(19) United States
(12) Patent Application Publication
(10) Pub. No.: US 2026/0103667 A1
(43) Pub. Date: Apr. 16, 2026

(54) IN VITRO MICROFLUIDIC SPHEROID-ASSOCIATED ANGIOGENESIS VASCULARIZATION SYSTEM AND METHOD FOR MAKING THE SAME

(71) Applicant: University of Maryland, College Park, College Park, MD (US)

(72) Inventors: Ian Smith, Silver Spring, MD (US); Kimberly Stroka, Davidsonville, MD (US); Sai Pranav Majeti Venkata, Rockville, MD (US); Autumn Hengen, Crofton, MD (US)

(21) Appl. No.: 19/355,089

(22) Filed: Oct. 10, 2025

Related U.S. Application Data
(60) Provisional application No. 63/706,185, filed on Oct. 11, 2024.

(51) Int. Cl. C12M 3/06; C12M 1/00; C12N 5/071; C12N 5/09

(52) U.S. Cl. CPC C12M 23/16; C12M 23/22; C12N 5/069; C12N 5/0693

(57) ABSTRACT

An in vitro microfluidic spheroid angiogenic vascularization system includes an optically transparent base; a reservoir adhered to the optically transparent base; a first media reservoir opening disposed along an external perimeter of the reservoir configured for receiving media; a first spheroid inlet configured to receive a cancer spheroid; a first cell inlet configured to receive an endothelial cell; a divider posed between the first spheroid inlet and the first cell inlet; a spheroid channel configured to transport the cancer spheroid from the first spheroid inlet to a central observation cavity; a cell channel configured to transport the endothelial cell from the first cell inlet to the central observation cavity; an outlet disposed along an end of the spheroid channel opposing the first spheroid inlet; and the central observation cavity filled with a volume of collagen into which the spheroid channel and the cell channel open.

### CROSS REFERENCE TO RELATED APPLICATIONS

The present application claims the benefit of and priority to U.S. Provisional Patent Application Serial Number 63/706,185 filed October 11, 2024 the entire content of which is incorporated herein in its entirety.

### GOVERNMENT SUPPORT

This invention was made with government support under R35 GM142838 awarded by the National Institutes of Health. The government has certain rights in the invention.

### TECHNICAL FIELD

The present disclosure relates to an in vitro microfluidic spheroid angiogenic vascularization system. More specifically, an aspect of the present disclosure provides systems and methods for in vitro observation of metastatic initiation. Further, an aspect of the present disclosure provides methods for identifying whether a test agent inhibits or stimulates angiogenesis and/or whether the test agent inhibits or stimulates metastatic activity.

### BACKGROUND

Metastasis remains the leading cause of cancer-related deaths, yet very few traditional chemotherapy systems focus on preventing spreading. Most of what is known regarding cancer metastasis is derived from retrospective studies, that is, studies that are conducted after the cancer had already spread throughout the body. However, understanding the initial metastatic steps are essential for developing essential drugs to aid in counteracting them. Tumor vascularization, in particular, is an essential facet of cancer development that involves providing oxygen and nutrients to proliferating cancer cells, while acting as a highway for cancer dissemination. Tumor cells actively recruit the formation of new blood vessels through the release of different angiogenic activators. Upon reception of these factors, endothelial cells (ECs) lining an endothelial vessel will invade towards the tumor, resulting in neovascularization. Simultaneously, cancer cells will dissociate from the tumor and migrate towards these endothelial vessels where they can intravasate and metastasize to distal sites. Traditionally, this relationship in metastatic initiation is investigated in vivo, particularly within mice, rats, and zebrafish. However, these models can lack relevance to human systems, prevent a real-time observation of mechanisms at play, and produce confounding results due to heterogeneity among animals. The actual translatability of cancer-based clinical trials from mice to humans is only a 10% success rate. Federal agencies such as the National Institutes of Health (“NIH”) and the Food and Drug Administration (“FDA”) have recognized the many shortcomings in the use of these animal models and have changed policies to encourage the use of in vitro models to recapitulate tumor microenvironments (TME) for pre-clinical testing. However, many of the traditional in vitro models oversimplify the relationship between tumors and their recruited ECs thus, hindering mechanistic translatability to in vivo models.

Accordingly, there is interest in an in vitro microfluidic spheroid angiogenic vascularization system that can replicate the cues associated with tumor vascularization and intravasation.

### SUMMARY

An aspect of the present disclosure provides an in vitro microfluidic spheroid angiogenic vascularization system. The in vitro microfluidic spheroid angiogenic vascularization system includes an optically transparent base and a reservoir adhered to the optically transparent base. The reservoir further includes a first media reservoir opening disposed along an external perimeter of the reservoir configured for receiving media; a first spheroid inlet configured to receive a cancer spheroid; a first cell inlet configured to receive an endothelial cell, wherein the first cell inlet is in fluid communication with the first media reservoir opening; a divider posed between the first spheroid inlet and the first cell inlet; a spheroid channel configured to transport the cancer spheroid from the first spheroid inlet to a central observation cavity; a cell channel configured to transport the endothelial cell from the first cell inlet to the central observation cavity; an outlet disposed along an end of the spheroid channel opposing the first spheroid inlet; and the central observation cavity filled with a volume of collagen into which the spheroid channel and the cell channel open.

In another aspect of the present disclosure, the reservoir may be adhered to the optically transparent base using a biocompatible adhesive.

In another aspect of the present disclosure, the biocompatible adhesive may be a silicone sealant.

In yet another aspect of the present disclosure, the reservoir may be a polydimethylsiloxane based negative.

In another aspect of the present disclosure, the in vitro microfluidic spheroid angiogenic vascularization system may be further comprised of a second media reservoir opening disposed along an external perimeter of the reservoir opposing the first media reservoir opening.

In another aspect of the present disclosure, the first spheroid inlet may be about 1mm wide.

In another aspect of the present disclosure, the first spheroid inlet may be comprised of rounded perimeter walls.

In yet another aspect of the present disclosure, a spacing between the first spheroid inlet and the first cell inlet measured across the divider may be between about 250 micrometers and about 1000 micrometers.

In another aspect of the present disclosure, fluid communication between the first cell inlet and the first media reservoir opening may be achieved using a biopsy punch to remove a portion of the reservoir.

In another aspect of the present disclosure, the removed portion of the reservoir may plug the first cell inlet to prevent endothelial cell outflow.

In yet another aspect of the present disclosure, the first cell inlet may be comprised of rounded perimeter walls.

In another aspect of the present disclosure, the in vitro microfluidic spheroid angiogenic vascularization system may be further comprised of a second cell inlet disposed along an end of the cell channel opposing the first cell inlet.

In another aspect of the present disclosure, the spheroid channel and cell channel may run parallelly.

In yet another aspect of the present disclosure, the central observation cavity may be entirely filled with collagen.

An aspect of the present disclosure provides a method for producing an in vitro microfluidic spheroid angiogenic vascularization system. The method includes disposing a reservoir in a media stored within a media reservoir, wherein the media is configured to enter the reservoir through a first reservoir opening disposed along an external perimeter of the reservoir. The method further includes filling a central observation cavity of the reservoir with a volume of collagen; depositing a cancer spheroid in a first spheroid inlet; depositing an endothelial cell in a first cell inlet, wherein the first cell inlet is in fluid communication with a first media reservoir opening; transporting the cancer spheroid through a spheroid channel to the central observation cavity; transporting the endothelial cell through a cell channel to the central observation cavity; and culturing the cancer spheroid and the endothelial cell in an incubator.

In another aspect of the present disclosure, the method may further include achieving fluid communication between the first cell inlet and the first media reservoir opening using a biopsy punch to remove a portion of the reservoir.

In another aspect of the present disclosure, the method may further include plugging the first cell inlet with the removed portion of the reservoir to prevent endothelial cell outflow.

In yet another aspect of the present disclosure, the method may further include disposing the incubator on a tilting table.

An aspect of the present disclosure provides a method of identifying whether a test agent inhibits or stimulates angiogenesis. The method comprises introducing a test agent into an in vitro microfluidic spheroid angiogenic vascularization system as described herein; maintaining the in vitro microfluidic spheroid angiogenic vascularization system under culture conditions; and determining whether the test agent inhibits or stimulates angiogenesis, wherein if the addition of the test agent to a medium stimulates angiogenic sprouting towards an angiogenic cocktail then the test agent is a stimulator of angiogenesis and wherein if the addition of the test agent inhibits angiogenic sprouting towards an angiogenic cocktail then the test agent is an inhibitor of angiogenesis. In such assays, the results are compared to controls where no test agent is added.

An aspect of the present disclosure provides a method of identifying whether a test agent inhibits or stimulates metastatic activity. The method comprises introducing a test agent into an in vitro microfluidic spheroid angiogenic vascularization system as described herein; maintaining the in vitro microfluidic spheroid angiogenic vascularization system under culture conditions; and determining whether the test agent inhibits or stimulates metastatic activity, wherein if the addition of the test agent stimulates cancerous invasion protrusions polarized towards an endothelial vessel then the test agent is a stimulator of metastatic activity and wherein if the addition of the test agent inhibits cancerous invasion protrusions polarized towards an endothelial vessel then the test agent is an inhibitor of metastatic activity. In such assays, the results are compared to controls where no test agent is added.

Further details and aspects of the present disclosure are described in more detail below with reference to the appended figures.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying figures of which:

FIG. 1 is an illustrative example of an in vitro microfluidic spheroid angiogenic vascularization system, in accordance with aspects of the present disclosure;

FIG. 2A is an illustrative example of the in vitro microfluidic spheroid angiogenic vascularization system of FIG. 1 wherein the in vitro microfluidic spheroid angiogenic vascularization system is placed in a 12-well media reservoir, in accordance with aspects of the present disclosure;

FIG. 2B is a cross-section view of the in vitro microfluidic spheroid angiogenic vascularization system of FIG. 1, in accordance with aspects of the present disclosure;

FIG. 2C is an illustrative example of a 12-well media reservoir, in accordance with aspects of the present disclosure;

FIG. 2D is a three-dimensional view of the in vitro microfluidic spheroid angiogenic vascularization system of FIG. 1, in accordance with aspects of the present disclosure;

FIG. 3A is microscopic image of an angiogenic sprouting towards an angiogenic cocktail, in accordance with aspects of the present disclosure;

FIG. 3B is a zoomed out microscopic image of the sprouts of FIG. 3A, in accordance with aspects of the present disclosure;

FIG. 3C is a diagram of sprout distance traveled, in accordance with aspects of the present disclosure;

FIG. 3D is a microscopic image of a spheroid invasion with chemokine, in accordance with aspects of the present disclosure;

FIG. 3E is a microscopic image of a spheroid invasion without chemokine, in accordance with aspects of the present disclosure;

FIG. 4A is a microscopic image demonstrating invasion in response to collagen density, in accordance with aspects of the present disclosure;

FIG. 4B is a diagram of sprout invasion distance, in accordance with aspects of the present disclosure;

FIG. 4C is a microscopic image of MDA-MB-231 invasion with no endothelial cell vascularization, in accordance with aspects of the present disclosure;

FIG. 4D is a microscopic image of no MCF10A invasion with endothelial cell vascularization, in accordance with aspects of the present disclosure;

FIG. 4E is a microscopic image demonstrating angiogenic sprouting in response to distance, in accordance with aspects of the present disclosure;

FIG. 5A is a microscopic image of an MDA-MB-231 spheroid invading towards an endothelial vessel, in accordance with aspects of the present disclosure;

FIG. 5B is a microscopic image of MDA-MB-231 cells in the endothelial vessel, in accordance with aspects of the present disclosure.

FIG. 6A is representative images of culturing of cells with and without Taxol for 6 days, in accordance with aspects of the present disclosure;

FIG. 6B is a representative image of spheroid invasion distance following Taxol treatment, in accordance with aspects of the present disclosure;

FIG. 6C is a representative image following 3 days of continuous culture in the absence of therapeutic intervention, in accordance with aspects of the present disclosure; and

FIG. 6D is a zoom-in image of the endothelial vessel with GFP-tagged cancer cells, in accordance with aspects of the present disclosure.

### DETAILED DESCRIPTION

The present disclosure relates to an in vitro microfluidic spheroid angiogenic vascularization system. Aspects of the present disclosure are described in detail with reference to the figures wherein like reference numerals identify similar or identical elements.

Although the present disclosure will be described in terms of specific aspects and examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary aspects illustrated in the figures, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

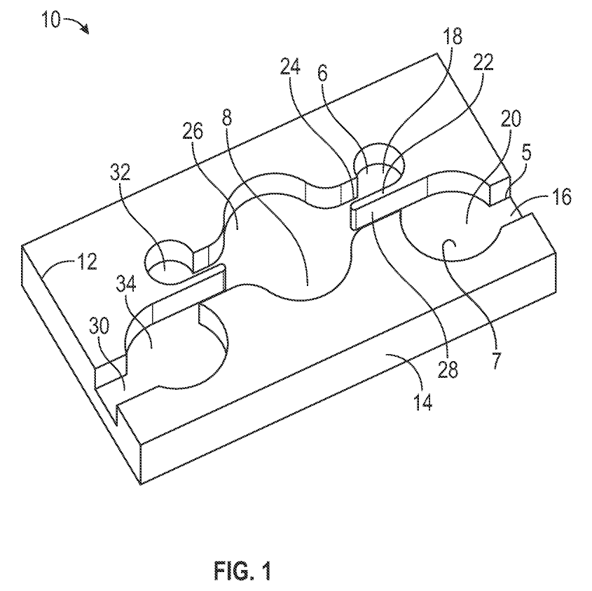

Referring to FIG. 1, an illustrative example of an in vitro microfluidic spheroid angiogenic vascularization system 10 is shown. The in vitro microfluidic spheroid angiogenic vascularization system 10 includes an optically transparent base 12 and a reservoir 14 (e.g., polymer-based, glass, silicate-based, hydrogel-based) adhered to the optically transparent base 12. The reservoir 14 may be adhered to the optically transparent base 12 using a biocompatible adhesive such as, but not limited to, silicone sealant, rubber, acrylate, polyurethane gels, cyanoacrylates, or medical-grade epoxies. In certain embodiments, the adhesive may include pressure sensitive adhesives, hydrogel adhesives, or bioresorbable adhesives that allow selective detachment and reattachment of the reservoir 14 to the optically transparent base 12. The reservoir 14 may be irreversibly adhered to the optically transparent base 12 to allow for repeated use of the in vitro microfluidic spheroid angiogenic vascularization system 10 after testing and following washing and sterilization procedures such as autoclaving, ultraviolet exposure, chemical sterilants, or plasma treatment depending on the selected materials. Adhering the reservoir 14 to the optically transparent base 12 allows for visualization of cellular dynamics within the in vitro microfluidic spheroid angiogenic vascularization system 10 using confocal microscopes, compound microscopes, phase-contrast microscopes, scanning electron microscopes, multiphoton microscopes, and optical coherence tomography. The optically transparent base 12 may be a glass coverslip of varying dimensions such as 18 millimeters in diameter, or other transparent substrates such as quartz, sapphire, polymethyl methacrylate, polycarbonate, cyclic olefin copolymer, or transparent conductive oxides such as indium tin oxide coated glass to allow integration with electrical stimulation and sensing. The optically transparent base 12 may also be patterned with microelectrodes, nano-textured surfaces, or biosensors for simultaneous imaging and electrical or biochemical measurement of cellular responses.

In various embodiments, the reservoir 14 may include synthetic or natural polymers. The reservoir 14 may include silicone polymers such as a polydimethylsiloxane based negative, a methyl vinyl silicone monomer unit, or a methyl hydrogen siloxane unit. Other embodiments may include polyethylene glycol diacrylate, polylactic acid, polycaprolactone, polyhydroxyethylmethacrylate, thermoplastic elastomers, chitosan, gelatin methacrylate, or composite hydrogels engineered for mechanical stability and controlled permeability. Silicone polymers offer low surface energy and high flexibility coefficients, providing water repellency, heat stability, and chemical attack resistance. Polydimethylsiloxane demonstrates a cost-effective nature, biocompatibility, chemical inertness, reversible deformability, oxygen permeability, and optical clarity. In embodiments, the reservoir 14 may be formed from non-polymeric materials such as borosilicate glass, fused silica, cyclic olefin copolymer (COC), polymethyl methacrylate (PMMA), or biocompatible resin composites fabricated by precision micromachining or additive manufacturing, thereby offering enhanced optical properties, chemical resistance, or structural rigidity depending on experimental requirements. The reservoir 14 may be of varying heights such as 2 millimeters or more and may be engineered to form modular stackable layers to allow for vertical integration with additional fluidic systems or organ-on-chip assemblies. The reservoir 14 may be injection molded, cast, or 3D printed, thereby supporting scalable manufacturing.

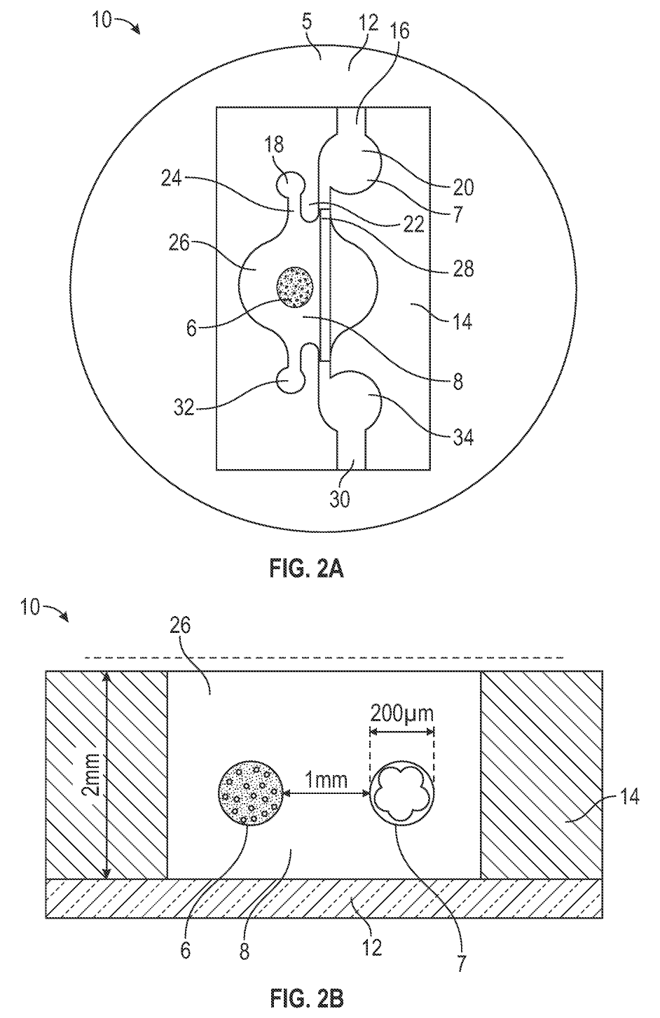

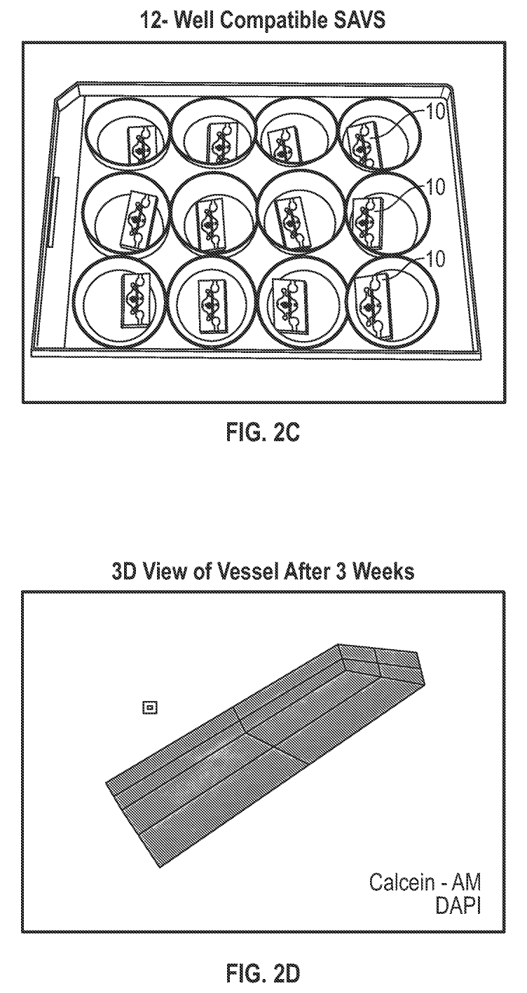

The reservoir 14 includes a first media reservoir opening 16 disposed along an external perimeter of the reservoir 14 configured for receiving media 5. The in vitro microfluidic spheroid angiogenic vascularization system 10 may be placed in a media reservoir to provide a constant supply of fresh media 5. The media reservoir may be formed, for example, by depositing media 5 in a cell culture plate well of varying dimensions such as a 12-well plate (FIG. 2C), 24-well plate, or 96-well plate. Once the cell culture plate well is filled with the media 5, the in vitro microfluidic spheroid angiogenic vascularization system 10 may be placed into the media reservoir such that a bottom surface of the optically transparent base 12 is disposed upon a top surface of the media reservoir and the first media reservoir opening 16 of the reservoir 14 is in fluid communication with the media 5. The media 5 may include Dulbecco’s Modified Eagle’s Medium, RPMI 1640, endothelial growth medium, neurobasal medium, serum-free chemically defined media, media 5 supplemented with patient-derived serum, organoid expansion media, or specialized formulations for stem cells, immune cells, hepatocytes, neurons, cardiac cells, or engineered microbial consortia. In certain embodiments, growth factors, cytokines, chemokines, hormones, drugs, nanoparticles, or viral vectors may be introduced into the media 5 to study dynamic responses. In embodiments, a second media reservoir opening 30 may be disposed along an end of the external perimeter of the reservoir 14 opposing the placement of the first media reservoir opening 16 to allow perfusion driven by gravity, pumps, or tilting tables. Additional reservoir openings may allow branching or parallel media supply lines for multiplexed experiments or controlled gradient generation. In certain embodiments, the reservoir 14 may be formed without one or both of the first media reservoir opening 16 and the second media reservoir opening 30, for example, in static culture configurations or when media 5 is manually exchanged rather than perfused.

The reservoir 14 further includes a first spheroid inlet 18 configured to receive a cancer spheroid 6, a first cell inlet 20 configured to receive an endothelial cell 7 (e.g., 250000 individual endothelial cells 7), and a divider 22 disposed between the first spheroid inlet 18 and the first cell inlet 20. The first spheroid inlet 18 may include rounded perimeter walls to allow efficient movement of spheroids such as cancer spheroids 6, stem cell aggregates, neural spheroids, pancreatic islets, hepatic organoids, embryoid bodies, or multicellular microbial colonies. Rectangular shapes or sharp corners may serve as fracture points and create nooks in which spheroids could become stuck, so rounded shapes are preferable. The first spheroid inlet 18 may be of varying dimensions but a width of approximately 1 millimeter is effective in allowing application of back pressure to force the cancer spheroid 6 or other spheroids into the in vitro microfluidic spheroid angiogenic vascularization system 10. In certain embodiments, an outlet 32 may be disposed along an end of a spheroid channel 24 opposing the placement of the first spheroid inlet 18. The spheroid channel 24 is configured to transport the cancer spheroid 6 or other spheroids from the first spheroid inlet 18 to a central observation cavity 26. The spheroids may be suspended in collagen 8 or any other basement membrane protein such as laminins, nidogens, heparan sulfate proteoglycans, perlecan, agrin, or synthetic analogues. Other extracellular matrices may include Matrigel®, fibrin, alginate, hyaluronic acid, decellularized tissue-derived matrices, or polyethylene glycol based hydrogels engineered with adhesive ligands or tunable stiffness. The spheroid channel 24 may be designed to accommodate spheroids of varying sizes, from hundreds of micrometers to millimeters, allowing scaling to organoid-level constructs.

The first cell inlet 20 may be in fluid communication with the first media reservoir opening 16 to maintain viability of cells such as endothelial cells 7 introduced into the central observation cavity 26. Other possible cell types introduced through the first cell inlet 20 include immune cells such as T cells, macrophages, or dendritic cells, stromal fibroblasts, mesenchymal stem cells, neurons, cardiomyocytes, hepatocytes, epithelial cells, or engineered cell lines. A biopsy punch may be used to remove a portion of the reservoir 14 encasing the external perimeter of the first cell inlet 20 prior to adhesion of the reservoir 14 to the optically transparent base 12. The removed portion may then be reinserted to create a plug to prevent cell outflow during seeding. Following adhesion of the endothelial cells 7 or other cells within the central observation cavity 26, the removed portion may be detached, unplugging the first cell inlet 20. The first cell inlet 20 may include rounded perimeter walls to avoid fracture points and facilitate efficient transport of endothelial cells 7. The first cell inlet 20 may be larger in diameter than the first spheroid inlet 18 because single cells may be transported with negative pressure driven flow rather than applied back pressure. In certain embodiments, a second cell inlet 34 may be disposed along an end of a cell channel 28 opposing the placement of the first cell inlet 20. The cell channel 28 is configured to transport endothelial cells 7 or other selected cell types from the first cell inlet 20 to the central observation cavity 26. The spheroid channel 24 and the cell channel 28 may run parallel along opposing sides of the divider 22. This configuration places the cancer spheroid 6 and the endothelial cells 7 in very close proximity within the collagen 8 environment, isolated from direct contact with artificial walls or supports, allowing study of spheroid-to-vessel, vessel-to-vessel, or multi-tissue interactions without non-physiological structures affecting outcomes. Additional dividers 22 or channel geometries may be implemented to control proximity, angle of approach, or gradient exposure of the vessels. In certain embodiments, the spacing between the first spheroid inlet 18 and the first cell inlet 20 measured across a divider 22 is selected from about 250 micrometers, about 500 micrometers, about 750 micrometers, or about 1000 micrometers, thereby allowing control of proximity and interaction timing between the cancer spheroid 6 and the endothelial cells 7.

The spheroid channel 24 may be engineered with variable lengths, widths, or curvatures to control the time required for the cancer spheroid 6 or other spheroids to enter the central observation cavity 26. The cell channel 28 may similarly be configured with microconstrictions or expansions that simulate vascular narrowing or dilation, thereby providing physiologically relevant flow resistance. Both the spheroid channel 24 and the cell channel 28 may be coated with extracellular matrix proteins, polysaccharides, or synthetic ligands to promote adhesion or migration of the cancer spheroid 6 or the endothelial cells 7. In some embodiments, the divider 22 positioned between the first spheroid inlet 18 and the first cell inlet 20 may be constructed of the same material as the reservoir 14, while in other embodiments it may include hydrogel or microporous membranes that allow selective diffusion of signaling molecules but prevent direct cell mixing before entry into the central observation cavity 26. The divider 22 may be straight, angled, curved, or branched depending on experimental design, allowing for studies of variable distances and orientations between the two input structures. Thus, the spheroid channel 24 and the cell channel 28 may serve as vessel-like conduits within the reservoir 14 for transporting the respective cancer spheroid 6 and endothelial cells 7 toward the central observation cavity 26.

The outlet 32 may be disposed at an opposite end of the spheroid channel 24, allowing loading of the cancer spheroid 6 or other spheroids from either side. This design may facilitate bidirectional studies, where spheroids introduced from opposite inlets migrate toward each other within the central observation cavity 26. Similarly, the second cell inlet 34 may be disposed at an opposite end of the cell channel 28, allowing for loading of endothelial cells 7 or other cells from either side. This configuration may allow parallel or converging vessel growth studies. The outlet 32 and second cell inlet 34 may be capped, plugged, or connected to secondary reservoirs to control flow direction. In certain embodiments, the outlet 32 or second cell inlet 34 may serve as outlets for waste removal, spent media 5, or effluent containing detached cells or extracellular vesicles for downstream analysis.

The central observation cavity 26 into which the spheroid channel 24 and the cell channel 28 open is filled with a volume of collagen 8. Collagen 8 may be added and polymerized across smaller doses in sequential layers to prevent capillary action from drawing excess collagen 8 into the first spheroid inlet 18 and first cell inlet 20. If collagen 8 accumulates in either inlet (18, 20), removal may be necessary prior to the addition of the cancer spheroid 6 or endothelial cells 7, as collagen 8 may obstruct their transport. The central observation cavity 26 may be entirely filled with collagen 8, as collagen 8 contracts during culture and an excess ensures maintenance of structural integrity. Alternative matrices may be used in place of collagen 8, such as polyethylene glycol based gels, fibrin gels, hyaluronic acid, agarose, synthetic peptide gels, or decellularized matrices. The central observation cavity 26 may also be filled with layered or gradient materials to mimic tissue stiffness gradients, oxygen gradients, or chemokine gradients. Prior to addition of needles such as subcutaneous needles, acupuncture needles, microinjection needles, or robotic pipette tips carrying the cancer spheroid 6 or endothelial cells 7, the needles may be treated with ethanol and surfactant solutions such as Pluronic® to prevent sticking to collagen 8.

The collagen 8 present in the central observation cavity 26 may be selected in different types such as type I collagen, type IV collagen, or mixtures thereof. Collagen 8 concentration may be varied to control stiffness, porosity, and fiber alignment. Collagen 8 may be crosslinked using chemical or enzymatic methods to modify degradation rates. In some embodiments, collagen 8 may be mixed with additional extracellular matrix components such as fibronectin, vitronectin, elastin, or glycosaminoglycans to provide specific cues for cell adhesion and migration. In other embodiments, collagen 8 may be replaced with engineered hydrogels whose stiffness can be dynamically altered by light, temperature, or chemical triggers, allowing studies of how mechanical changes influence invasion or vascularization. Collagen 8 may also be doped with fluorescent dyes, nanoparticles, or biosensors to allow visualization of matrix remodeling, protease activity, or gradient diffusion.

The central observation cavity 26 may be fabricated with circular, oval, or irregular geometries to mimic different anatomical sites and to allow for customized flow profiles and diffusion gradients. The central observation cavity 26 may include embedded markers, fiducial grids, or microstructured topographies to aid in imaging alignment and quantitative analysis of cellular invasion, vessel sprouting, and intravasation. In certain embodiments, the central observation cavity 26 may include ports for insertion of sensors that measure oxygen, pH, glucose, lactate, or cytokine concentrations in real time.

In embodiments, the first media reservoir opening 16 and the second media reservoir opening 30 may be connected by external tubing to pumps or to gravity-driven reservoirs to allow for dynamic perfusion of the media 5. In some arrangements, the in vitro microfluidic spheroid angiogenic vascularization system 10 is cultured on a tilting table (not shown) that provides periodic angular modulation to drive media 5 exchange between the first media reservoir opening 16 and the second media reservoir opening 30, thereby promoting continuous nutrient replenishment and waste removal. The media 5 may be continuously recirculated, exchanged periodically, or supplemented with defined pulses of growth factors, chemokines, or drugs to model acute exposures. In certain embodiments, additional reservoir openings may be included to introduce multiple media 5 streams, thereby generating competing or overlapping gradients across the central observation cavity 26. This arrangement may allow simultaneous study of angiogenesis, chemotaxis, and drug diffusion.

Standard techniques for cell culture, e.g., media, temperature, etc., may be used for culturing cells of the system. Such techniques and procedures may be generally performed according to conventional methods well known in the art and are generally designed to create an environment for optimal cell growth. Techniques for cell growth will depend upon the choice of cells to be cultured in the provided cell culture system. The media 5 may include supplements such as fetal bovine serum, human serum, platelet-rich plasma, synthetic sera, or purified growth factors. Antibiotics, antifungals, or antivirals may be included to maintain sterility or to investigate antimicrobial resistance. The media 5 may also carry nanoparticles, extracellular vesicles, viral vectors, or gene-editing complexes such as CRISPR-Cas to evaluate delivery and uptake in the context of multicellular structures.

The optically transparent base 12 in combination with the reservoir 14 may allow imaging at single-cell resolution in real time. The proximity of the spheroid channel 24 and the cell channel 28 within the basement membrane environment of the central observation cavity 26 ensures that the cancer spheroid 6 and the endothelial cells 7 interact in a physiologically relevant manner without interference from artificial walls or substrates. This allows the in vitro microfluidic spheroid angiogenic vascularization system 10 to replicate processes such as angiogenic sprouting, cancer cell invasion, intravasation, extravasation, immune cell trafficking, organoid-to-organoid communication, viral dissemination, and drug response in a controlled and observable format.

The in vitro microfluidic spheroid angiogenic vascularization system 10 may be scaled into arrays, with multiple reservoirs 14 fabricated on a single optically transparent base 12. Each unit may include its own first media reservoir opening 16, second media reservoir opening 30, first spheroid inlet 18, outlet 32, first cell inlet 20, second cell inlet 34, spheroid channel 24, cell channel 28, divider 22, and central observation cavity 26 filled with collagen 8 or other matrices. Such arrays may allow high-throughput screening of drugs, toxins, or genetic perturbations. Automated systems may include robotic handling of media 5, automated loading of cancer spheroids 6 and endothelial cells 7, and integrated imaging platforms. Scale-up embodiments may connect multiple in vitro microfluidic spheroid angiogenic vascularization systems 10 in series or parallel to mimic tissue-to-tissue communication or organ-level physiology, such as modeling primary tumor sites and metastatic niches simultaneously.

Applications of the in vitro microfluidic spheroid angiogenic vascularization system 10 extend across oncology, regenerative medicine, immunology, neuroscience, virology, microbiology, toxicology, developmental biology, and environmental science. In oncology, the in vitro microfluidic spheroid angiogenic vascularization system 10 may be used to evaluate tumor angiogenesis, metastatic initiation, drug sensitivity, resistance development, and patient-specific responses using spheroids derived from patient biopsies. In regenerative medicine, the in vitro microfluidic spheroid angiogenic vascularization system 10 may study vascular integration of stem-cell derived tissues or evaluate biomaterials for tissue engineering. In immunology and virology, the in vitro microfluidic spheroid angiogenic vascularization system 10 may assess immune cell infiltration, viral infection pathways, or vaccine responses. In neuroscience, the in vitro microfluidic spheroid angiogenic vascularization system 10 may be adapted to study blood-brain barrier integrity, neurovascular coupling, or brain organoid vascularization. In microbiology, the in vitro microfluidic spheroid angiogenic vascularization system 10 may be used to examine host-pathogen interactions, microbial invasion, or polymicrobial communities within matrices. In toxicology and environmental science, the in vitro microfluidic spheroid angiogenic vascularization system 10 may assess pollutant effects, nanoparticle transport, or food and cosmetic ingredient safety without reliance on animal testing.

The in vitro microfluidic spheroid angiogenic vascularization system 10 establishes two vessels, defined by the spheroid channel 24 and the cell channel 28, positioned in very close proximity within the central observation cavity 26 that is filled with collagen 8 or other basement membrane-like material. Unlike platforms that rely on rigid scaffolds, posts, or non-physiological barriers to confine cells and matrices, this arrangement allows the cancer spheroid 6 introduced through the first spheroid inlet 18 and the endothelial cell 7 introduced through the first cell inlet 20 to interact within an environment that more closely resembles in vivo tissue structure. Because the divider 22 separates the entry of the two populations yet directs them toward the same central observation cavity 26, the in vitro microfluidic spheroid angiogenic vascularization system 10 permits precise control over spatial relationships, gradient formation, and timing of interaction without interference from artificial boundaries. The combination of the optically transparent base 12 and the reservoir 14 permits direct, high-resolution visualization of dynamic cellular processes, including angiogenic sprouting, spheroid invasion, and intravasation, under conditions that can be carefully tuned by modifying the media 5 delivered through the first media reservoir opening 16 and second media reservoir opening 30. The configuration of multiple inlets, including the outlet 32 and the second cell inlet 34, provides flexibility for unidirectional or bidirectional loading, co-culture, or gradient studies. By filling the central observation cavity 26 with collagen 8 or alternative extracellular matrices, the in vitro microfluidic spheroid angiogenic vascularization system 10 reproduces key biophysical and biochemical cues while avoiding collapse or detachment of the matrix from device walls. This design offers significant advantages for research and development because it allows observation of multicellular behaviors, supports reproducibility across experiments, and can be readily scaled into high-throughput arrays.

The structure of the in vitro microfluidic spheroid angiogenic vascularization system 10 is broadly adaptable, permitting its use not only in cancer biology but also in regenerative medicine, immunology, neuroscience, virology, microbiology, and toxicology, and it provides a platform that combines physiological accuracy with compatibility for imaging, automation, and diverse experimental conditions.

Accordingly, the present disclosure provides a tissue culture system that allows for the investigation of the spatio-temporal dynamics of multicellular interactions. In a specific embodiment, methods are provided for visualizing metastatic initiation mechanisms. As described below, the provided tissue culture system may be used in screening assays for identifying therapeutic agents that correct, or alleviate, one or more of the cell culture phenotypes associated with metastatic initiation techniques. Such phenotypes include, for example, endothelial vessel sprouting, angiogenesis, vessel sprouting tumor outgrowth, and tumor cell intravasation and extravasation.

An aspect of the present disclosure provides a method of identifying whether a test agent inhibits or stimulates angiogenesis. The method comprises introducing a test agent into an in vitro microfluidic spheroid angiogenic vascularization system 10 as described herein; maintaining the in vitro microfluidic spheroid angiogenic vascularization system 10 under culture conditions; and determining whether the test agent inhibits or stimulates angiogenesis, wherein if the addition of the test agent to media 5 stimulates angiogenic sprouting towards an angiogenic cocktail then the test agent is a stimulator of angiogenesis and wherein if the addition of the test agent inhibits angiogenic sprouting towards an angiogenic cocktail then the test agent is an inhibitor of angiogenesis. In such assays, the results are compared to controls where no test agent is added.

An aspect of the present disclosure provides a method of identifying whether a test agent inhibits or stimulates metastatic activity. The method comprises introducing a test agent into an in vitro microfluidic spheroid angiogenic vascularization system 10 as described herein; maintaining the in vitro microfluidic spheroid angiogenic vascularization system 10 under culture conditions; and determining whether the test agent inhibits or stimulates metastatic activity, wherein if the addition of the test agent stimulates cancerous invasion protrusions polarized towards an endothelial vessel then the test agent is a stimulator of metastatic activity and wherein if the addition of the test agent inhibits cancerous invasion protrusions polarized towards an endothelial vessel then the test agent is an inhibitor of metastatic activity. In such assays, the results are compared to controls where no test agent is added.

Exemplary test agents include, but are not limited to, peptides, such as soluble peptides, including but not limited to members of random peptide libraries, antibodies and antibody fragments and small organic or inorganic molecules. Appropriate test compounds can be contained in libraries, for example, synthetic or natural compounds in a combinatorial library. Numerous libraries are commercially available or can be readily produced; means for random and directed synthesis of a wide variety of organic compounds and biomolecules also are known. Alternatively, libraries of natural compounds in the form of bacterial, fungal, plant and animal extracts are available or can be readily produced. Such libraries are useful for the screening of a large number of different compounds for identification of therapeutic agents that inhibit cancer metastatic activity.

Referring now to FIGS. 2A-2D, a method for producing the in vitro microfluidic spheroid angiogenic vascularization system 10 is shown. The method includes disposing the reservoir 14 on the optically transparent base 12 and placing the system into media 5 stored within a cell culture plate well (e.g., the 12-well cell culture plate depicted in FIG. 2C) such that the second media reservoir opening 30 of the reservoir 14 is in fluid communication with the media 5. The media 5 is configured to enter the reservoir 14 through the first media reservoir opening 16 disposed along the external perimeter of the reservoir 14 thereby allowing for the constant flow of media 5 through the in vitro microfluidic spheroid angiogenic vascularization system 10 in a sterile in vitro environment that can be finely tuned to replicate various environmental factors (e.g., temperature gradients, pressure gradients) and easily observed through the use of the multitude of imaging modalities. The method further includes filling the central observation cavity 26 of the reservoir 14 with collagen 8 prior to depositing the cancer spheroid 6 in the first spheroid inlet 18 and the endothelial cell 7 in the first cell inlet 20 that is in fluid communication with the first media reservoir opening 16. The cancer spheroid 6 and the endothelial cell 7 are then transported through the respective spheroid channel 24 and cell channel 28 to the central observation cavity 26. The cancer spheroids 6 are added to the in vitro microfluidic spheroid angiogenic vascularization system 10 while suspended in collagen 8.

Once the cancer spheroid 6 and the endothelial cell 7 are present in the central observation cavity 26, the cancer spheroid 6 and the endothelial cell 7 are cultured in an incubator. The incubator creates an environment for cell growth and maintains a balance of temperature, humidity, and carbon dioxide levels, amongst other parameters, to ensure that the cancer spheroid 6 and the endothelial cell 7 are able to survive and flourish. This allows for in vitro observation of a replication of the cues associated with tumor vascularization and cancer spheroid 6 intravasation into endothelial vessels and, ultimately, the cellular phenomena associated with the advent of metastasis. More specifically, the in vitro microfluidic spheroid angiogenic vascularization system 10 may be utilized to demonstrate the effects of cancer detachment from a primary tumor, directed invasion through a basement membrane, and intravasation into an endothelial vessel. In various embodiments, the incubator may be disposed on a tilting table to promote constant flow of media 5 through the in vitro microfluidic spheroid angiogenic vascularization system 10 by adjusting the angular placement of the tilting table.

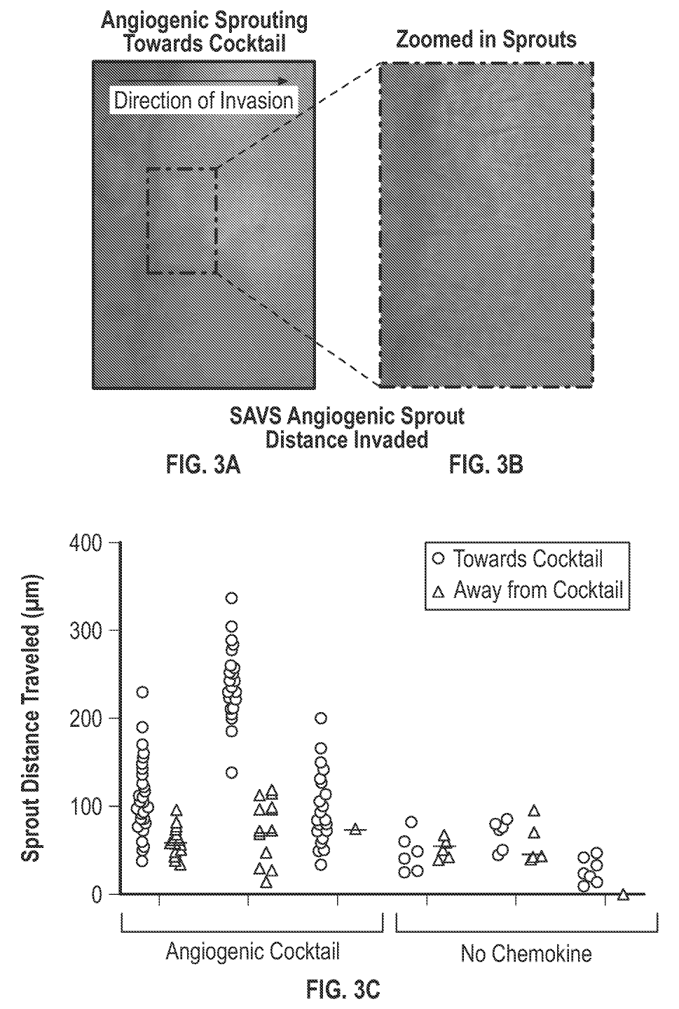

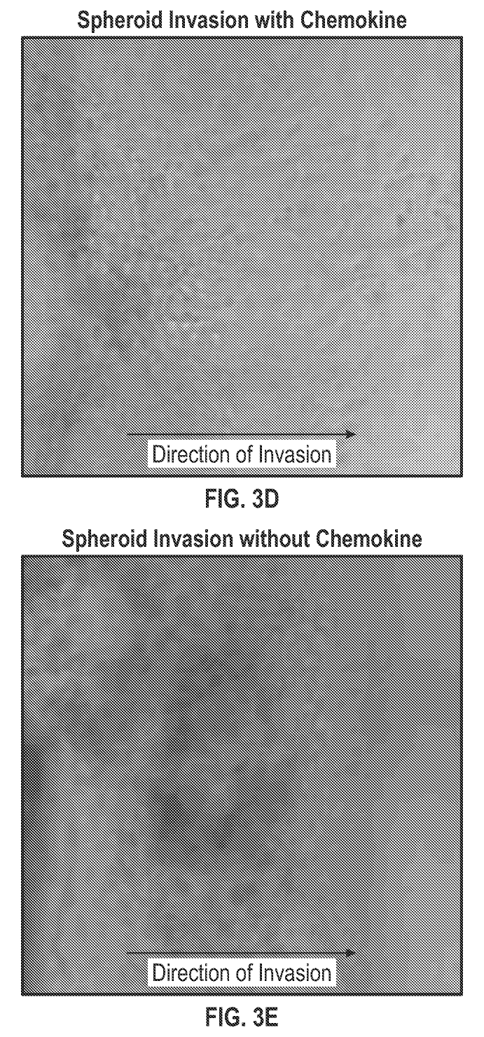

Referring to FIGS. 3A-3E, various microscopic images and diagrams related to angiogenic sprouting using chemokine are shown. Namely, FIG. 3C demonstrates sprout distance traveled towards and away from an angiogenic cocktail when chemokine is introduced to the system (FIG. 3D) and when chemokine is withheld (FIG. 3E). When cancer spheroids 6 and endothelial cells 7 are cultured independent of one another within the in vitro microfluidic spheroid angiogenic vascularization system 10, they exhibit significant increases in unilateral invasion in response to chemotactic gradients (FIG. 3C) and both cancer spheroids 6 and endothelial cells 7 invade unilaterally towards the direction of added chemokine (FIG. 3C).

An angiogenic nature of a test agent may be determined by introducing a test agent into the in vitro microfluidic spheroid angiogenic vascularization system 10 described herein, maintaining the in vitro microfluidic spheroid angiogenic vascularization system 10 under culture conditions, and determining whether the test agent inhibits or stimulates angiogenesis. This determination as to the test agent’s angiogenic nature, that is whether the test agent inhibits or stimulates angiogenesis, may be accomplished by observing if the addition of the test agent to media 5 stimulates angiogenic sprouting, the growth of new blood vessels from existing ones, towards an angiogenic cocktail, thereby indicating that the test agent is a stimulator of angiogenesis. However, if the addition of the test agent inhibits angiogenic sprouting towards an angiogenic cocktail, then the test agent is an inhibitor of angiogenesis. In such assays, the results are compared to controls where no test agent is added. In the validation of the in vitro microfluidic spheroid angiogenic vascularization system 10, initial tests are conducted to ensure that endothelial cells 7 within the device would invade towards their opposite structure (e.g., cancer spheroids 6). In order to achieve this, a chemokine, or any structure that causes endothelial cells 7 to invade and/or migrate, is placed in an opposite inlet, and thus an opposite channel, of a structure being observed. Following these tests, the endothelial cells 7 are cultured within the in vitro microfluidic spheroid angiogenic vascularization system 10 and a chemotherapeutic (e.g., Taxol) is added to the media 5 surrounding the system in the media reservoir that is connected to the first cell inlet 20 and the first spheroid inlet 18 by the cell channel 28 and the spheroid channel 24 punched through the system walls. Taxol has been shown to reduce the speed of endothelial cell 7 invasion, while also preventing cancer spheroid 6 proliferation.

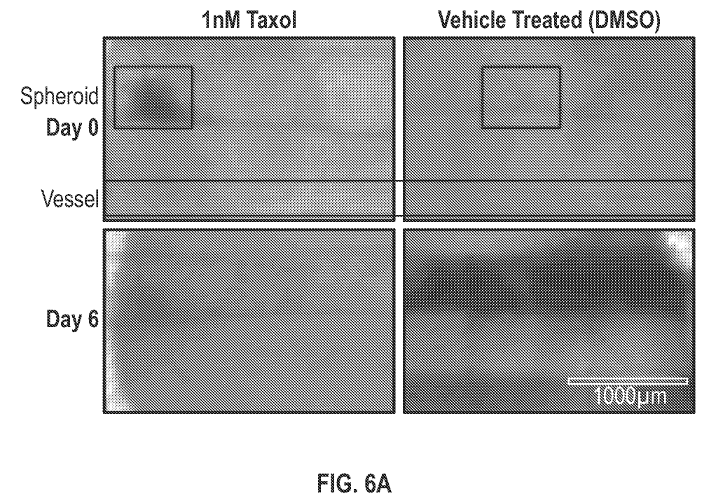

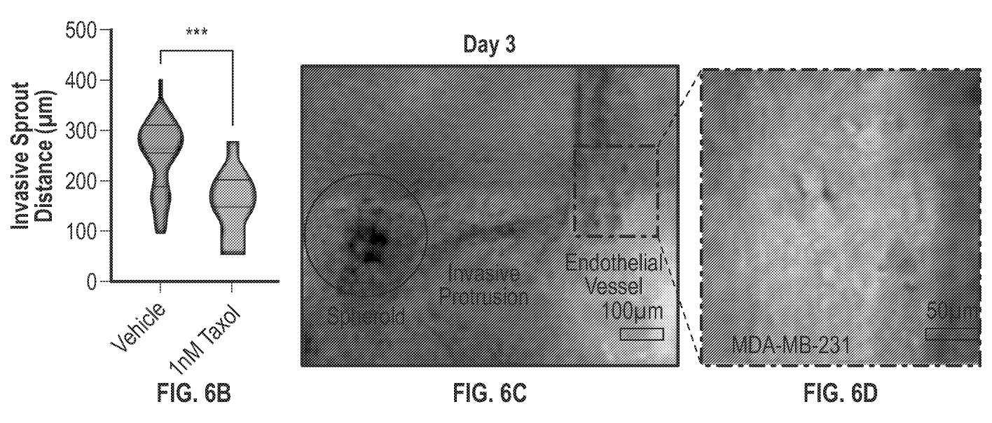

FIG. 6A is representative image of cultured endothelial cells 7 with and without Taxol for 6 days. FIG. 6B is a representative image of cancer spheroid 6 invasion distance following Taxol treatment. In the absence of therapeutic interventions, within three days of culture, GFP-tagged cancer cells can be identified in the endothelial vessel, indicating vascular intravasation (FIGS. 6C and D). FIG. 6C is a representative image following 3 days of continuous culture and FIG. 6D is a zoom-in image of the endothelial vessel with GFP-tagged cancer cells.

In some embodiments, the in vitro microfluidic spheroid angiogenic vascularization system 10 may be used in precision medicine to test for drugs that inhibit angiogenic activity in a patient. The test agent may be a chick chorioallantoic membrane (“CAM”) assay, a subcutaneous matrigel plug assay, an aortic ring assay, or a mouse metatarsal assay. The angiogenic cocktail includes a combination of factors that may trigger angiogenesis including, but not limited to, vascular endothelial growth factor-165 (“VEGF-165”), phorbol 12-myristate 13-acetate (“PMA”), monocyte chemoattractant protein-1 (“MCP-1”), and sphingosine-1-phosphate (“S1P”). Angiogenic factors are generally tested in low-serum or low-growth factor conditions.

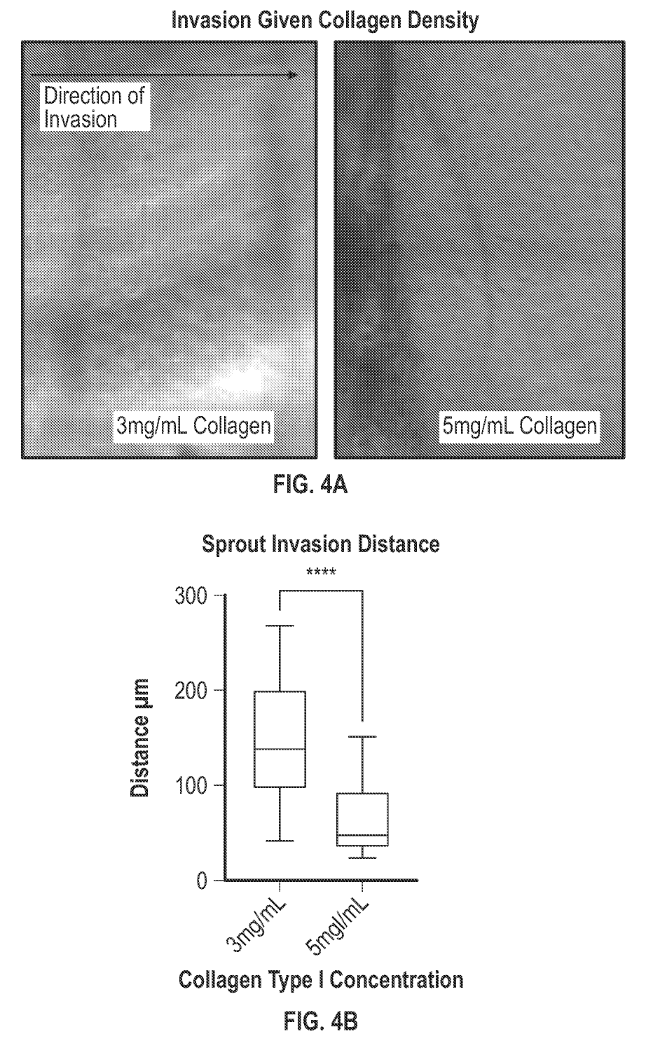

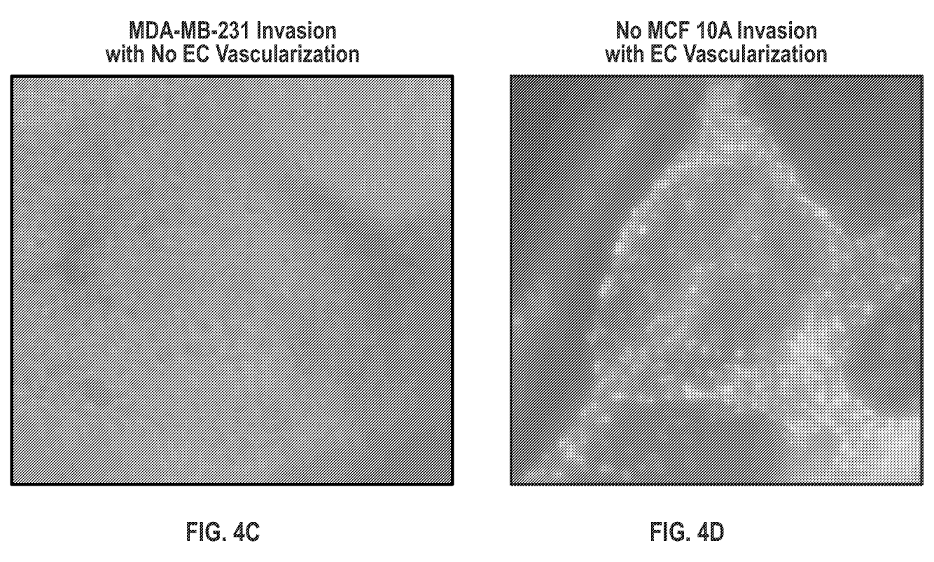

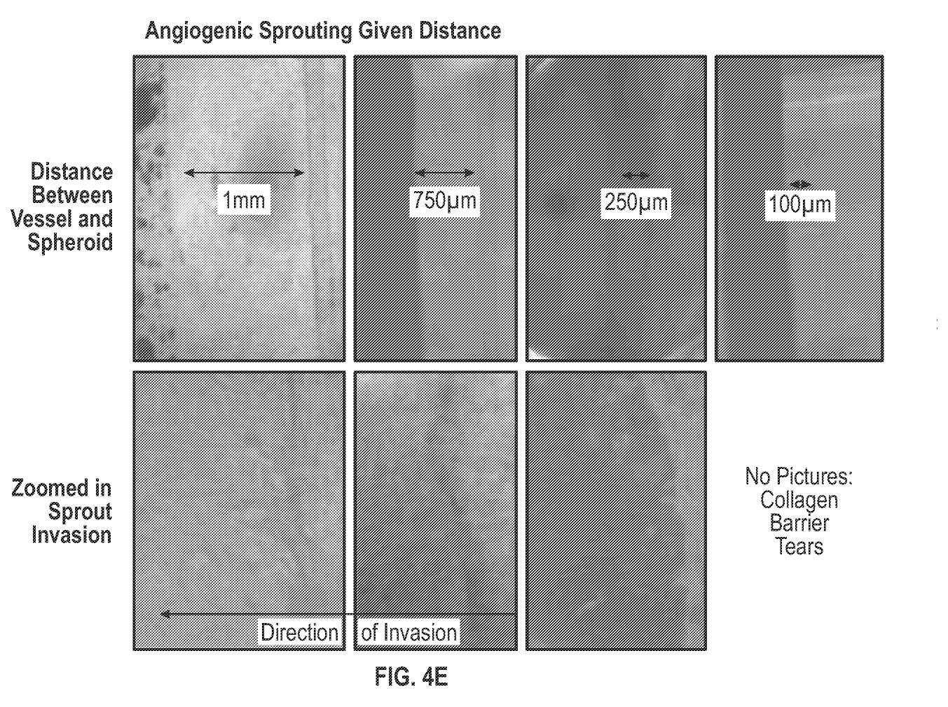

Referring now to FIGS. 4A-4E, various microscopic images and diagrams related to sprout invasion in response to collagen 8 density (FIG. 4A), MDA-MB-231 when there is no endothelial cell 7 vascularization (FIG. 4C), MCF10A when there is endothelial cell 7 vascularization (FIG. 4D), and a distance gradient between the cancer spheroid 6 and the endothelial cell 7 (FIG. 4E) are shown. More specifically, the microscopic images of FIGS. 4A and 4C-4E demonstrate: 1) cellular invasion decreases when the collagen 8 is polymerized at a higher density; 2) the invasion of the cancer spheroid 6 and the endothelial cell 7 are correlated positively to the metastatic potential of cells including the cancer spheroids 6; 3) the invasive potential of the endothelial cells 7 and the cancer spheroids 6 are inversely dependent on the distance between the two structures; and 4) treatment of cells with the chemotherapeutic Taxol as a means of inhibiting cell invasion and proliferation.

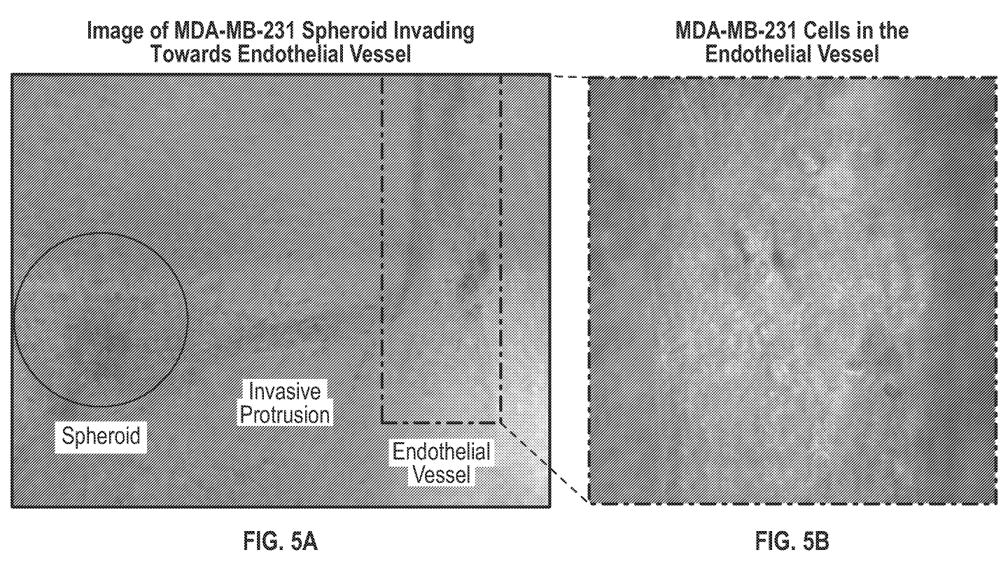

Referring to FIGS. 5A and 5B, microscopic images related to MDA-MB-231 cancer spheroid 6 (FIG. 5A) and endothelial cell 7 (FIG. 5B) invasion are shown. Culturing endothelial cells 7 with MDA-MB-231 cancer spheroids 6 causes defined cancerous invasion protrusions to form that are polarized towards the endothelial vessel (FIG. 5A), thereby indicating metastatic stimulation.

A metastatic nature of a test agent, that is whether the test agent inhibits or stimulates metastatic activity, may be determined by introducing a test agent into the in vitro microfluidic spheroid angiogenic vascularization system 10 described herein, maintaining the in vitro microfluidic spheroid angiogenic vascularization system 10 under culture conditions, and determining whether the test agent inhibits or stimulates metastatic activity. Metastatic activity may be determined by observing if the addition of the test agent stimulates cancerous invasion protrusions polarized towards an endothelial vessel, thereby indicating that the test agent is a stimulator of metastatic activity. However, if the addition of the test agent inhibits cancerous invasion protrusions polarized towards an endothelial vessel, then the test agent is an inhibitor of metastatic activity. In such assays, the results are compared to controls where no test agent is added. In some embodiments, the in vitro microfluidic spheroid angiogenic vascularization system 10 may be used in precision medicine through the use of a cancer spheroid 6 derived from a patient to determine whether particular drugs inhibit metastatic activity for the patient.

Certain aspects of the present disclosure may include some, all, or none of the above advantages and/or one or more other advantages readily apparent to those skilled in the art from the figures, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached figures are presented only to demonstrate certain examples of the disclosure. Other elements, steps, methods, and techniques that are insubstantially different from those described above and/or in the appended claims are also intended to be within the scope of the disclosure.

What is claimed is:

1. An in vitro microfluidic spheroid angiogenic vascularization system, comprising:
an optically transparent base; and
a reservoir adhered to the optically transparent base, the reservoir including:
a first media reservoir opening disposed along an external perimeter of the reservoir configured for receiving a media;
a first spheroid inlet configured to receive a cancer spheroid;
a first cell inlet configured to receive an endothelial cell, wherein the first cell inlet is in fluid communication with the first media reservoir opening;
a divider posed between the first spheroid inlet and the first cell inlet;
a spheroid channel configured to transport the cancer spheroid from the first spheroid inlet to a central observation cavity;
a cell channel configured to transport the endothelial cell from the first cell inlet to the central observation cavity;
an outlet disposed along an end of the spheroid channel opposing the first spheroid inlet; and
the central observation cavity filled with a volume of collagen into which the spheroid channel and the cell channel open.

2. The in vitro microfluidic spheroid angiogenic vascularization system of claim 1, wherein the reservoir is adhered to the optically transparent base using a biocompatible adhesive.

3. The in vitro microfluidic spheroid angiogenic vascularization system of claim 2, wherein the biocompatible adhesive is a silicone sealant.

4. The in vitro microfluidic spheroid angiogenic vascularization system of claim 1, wherein the reservoir is a polydimethylsiloxane based negative.

5. The in vitro microfluidic spheroid angiogenic vascularization system of claim 1, further comprising a second media reservoir opening disposed along an external perimeter of the reservoir opposing the first media reservoir opening.

6. The in vitro microfluidic spheroid angiogenic vascularization system of claim 1, wherein the first spheroid inlet is about 1mm wide.

7. The in vitro microfluidic spheroid angiogenic vascularization system of claim 1, wherein the first spheroid inlet is comprised of rounded perimeter walls.

8. The in vitro microfluidic spheroid angiogenic vascularization system of claim 1,
250250 micrometers and about 1000 micrometers.

9. The in vitro microfluidic spheroid angiogenic vascularization system of claim 1, wherein fluid communication between the first cell inlet and the first media reservoir opening is achieved using a biopsy punch to remove a portion of the reservoir.

10. The in vitro microfluidic spheroid angiogenic vascularization system of claim 9, wherein the removed portion of the reservoir plugs the first cell inlet to prevent endothelial cell outflow.

11. The in vitro microfluidic spheroid angiogenic vascularization system of claim 1, wherein the first cell inlet is comprised of rounded perimeter walls.

12. The in vitro microfluidic spheroid angiogenic vascularization system of claim 1, further comprising a second cell inlet disposed along an end of the cell channel opposing the first cell inlet.

13. The in vitro microfluidic spheroid angiogenic vascularization system of claim 1, wherein the spheroid channel and cell channel run parallelly.

14. The in vitro microfluidic spheroid angiogenic vascularization system of claim 1, wherein the central observation cavity is entirely filled with collagen.

15. A method for producing an in vitro microfluidic spheroid angiogenic vascularization system, the method comprising:
disposing a reservoir in a media stored within a media reservoir, wherein the media is configured to enter the reservoir through a first reservoir opening disposed along an external perimeter of the reservoir;
filling a central observation cavity of the reservoir with a volume of collagen;
depositing a cancer spheroid in a first spheroid inlet;
depositing an endothelial cell in a first cell inlet, wherein the first cell inlet is in fluid communication with a first media reservoir opening;
transporting the cancer spheroid through a spheroid channel to the central observation cavity;
transporting the endothelial cell through a cell channel to the central observation cavity; and
culturing the cancer spheroid and the endothelial cell in an incubator.

16. The method of claim 15, further comprising achieving fluid communication between the first cell inlet and the first media reservoir opening using a biopsy punch to remove a portion of the reservoir.

17. The method of claim 16, further comprising plugging the first cell inlet with the removed portion of the reservoir to prevent endothelial cell outflow.

18. The method of claim 15, further comprising disposing the incubator on a tilting table.

19. A method of identifying whether a test agent inhibits or stimulates angiogenesis, the method comprising:
introducing a test agent into the in vitro microfluidic spheroid angiogenic vascularization system of claim 1;
maintaining the in vitro microfluidic spheroid angiogenic vascularization system under culture conditions; and
determining whether the test agent inhibits or stimulates angiogenesis, wherein if the addition of the test agent to a medium stimulates angiogenic sprouting towards an angiogenic cocktail then the test agent is a stimulator of angiogenesis, and wherein if the addition of the test agent inhibits angiogenic sprouting towards an angiogenic cocktail then the test agent is an inhibitor of angiogenesis.

20. A method of identifying whether a test agent inhibits or stimulates metastatic activity, the method comprising:
introducing a test agent into the in vitro microfluidic spheroid angiogenic vascularization system of claim 1;
maintaining the in vitro microfluidic spheroid angiogenic vascularization system under culture conditions; and
determining whether the test agent inhibits or stimulates metastatic activity, wherein if the addition of the test agent stimulates cancerous invasion protrusions polarized towards an endothelial vessel then the test agent is a stimulator of metastatic activity, and wherein if the addition of the test agent inhibits cancerous invasion protrusions polarized towards an endothelial vessel then the test agent is an inhibitor of metastatic activity.
